SEQUENCE LISTING (1 sequence)

SEQ ID NO: 1 - protein, 10 aa, Homo sapiens
LFVDTDADTR

(19) United States
(12) Patent Application Publication
(10) Pub. No.: US 2020/0271653 A1
(43) Pub. Date: Aug. 27, 2020

(54) UCK2 Assay To Predict Cancer Therapy Response

(71) Applicant: NantOmics, LLC, Culver City, CA (US)

(72) Inventors: Dongyao YAN, Culver City, CA (US); Fabiola CECCHI, Potomac, MD (US); Todd HEMBROUGH, Gaithersburg, MD (US)

(21) Appl. No.: 16/647,911

(22) PCT Filed: Sep. 20, 2018
(86) PCT No.: PCT/US18/52084

§ 371 Date: Mar. 17, 2020

Related U.S. Application Data
(60) Provisional application No. 62/560,764, filed on Sep. 20, 2017.

(51) Int. Cl. G01N 33/574; G01N 33/68

(52) U.S. Cl. CPC G01N 33/57419; A61K 31/513; G01N 33/6848

(57) ABSTRACT

A method is provided for quantifying the UCK2 protein in biological samples that have been fixed in formalin by the method of Selected Reaction Monitoring (SRM) mass spectrometry and utilizing said quantitation of UCK2 to predict the therapeutic outcome of treating a colon cancer patient with the combinatorial FOLFOX (5-fluorouracil, folinic acid, and oxaliplatin) treatment regimen.

This application claims priority to U.S. Provisional Application No. 62/560,764, filed Sep. 20, 2017, the contents of which are hereby incorporated by reference in their entirety. This application also contains a sequence listing submitted electronically via EFS-web, which serves as both the paper copy and the computer readable form (CRF) and consists of a file entitled “SeqListing_3900_00761”, which was created on Sep. 19, 2018, which is 390 bytes in size, and which also is incorporated by reference in its entirety.

### INTRODUCTION

The level of protein expression of the UCK2 protein in patient tumor tissue is determined by quantitating a specified peptide derived from the full-length UCK2 protein. The specified peptide is detected using mass spectrometry-based Selected Reaction Monitoring (SRM, also referred to as Multiple Reaction Monitoring (MRM), and referred to herein as an SRM assay). An SRM assay is used to detect the presence and quantitatively measure the amount of a specified UCK2 fragment peptide, directly in cells procured from cancer patient tissue, such as, for example formalin fixed cancer tissue.

The quantitation is relative or absolute. When absolute quantitation is required the measured level of the specified UCK2 peptide is compared to a known amount of a labeled reference peptide having the same amino acid sequence as the measured peptide. The specified UCK2 peptide is unique to UCK2, and therefore one peptide molecule is derived from one protein molecule and the quantitative level of the peptide allows quantitation of the intact UCK2 protein. The measurement of UCK2 protein expression can be used for diagnosis of cancer, staging of the cancer, prognosis of cancer progression, predicting the likelihood of clinical response to various cancer treatments and therapies. The presently described method provides a specific UCK2 cutoff level such that, patients whose tumor cells express UCK2 at a level higher than the cutoff will have a significantly greater chance of longer overall survival when treated with the FOLFOX (combination 5-fluorouracil, folinic acid, and oxaliplatin) cancer therapy regimen than patients whose tumor cells express UCK2 at a level below the cutoff. In one embodiment the cutoff level is 319 amol/μg of total protein analyzed.

### SUMMARY OF THE INVENTION

What is provided is a method for measuring the level of the human UCK2 protein in a human biological sample of formalin-fixed tissue, comprising detecting and quantifying the amount of a UCK2 fragment peptide in a protein digest, such as a protease digest, prepared from the human biological sample using mass spectrometry; and calculating the level of UCK2 protein in the sample; where the UCK2 fragment peptide is SEQ ID NO:1, and where the level is a relative level or an absolute level. The protein digest may be fractionated prior to detecting and/or quantifying the amount of the UCK2 fragment peptide.

The tissue may be paraffin-embedded tissue, and may be obtained from a tumor. On one embodiment, quantifying the UCK2 fragment peptide involves comparing the amount of the UCK2 fragment peptide in one biological sample to the amount of the same UCK2 fragment peptide in a different and separate biological sample. In another embodiment, quantifying the UCK2 fragment peptide involves determining the amount of the UCK2 fragment peptide in a biological sample by comparison to an added internal standard peptide of known amount, where the UCK2 fragment peptide in the biological sample is compared to an internal standard peptide having the same amino acid sequence; and where the internal standard peptide is an isotopically labeled peptide. The isotopically labeled internal standard peptide may contain one or more heavy stable isotopes selected from ¹⁸O, ¹⁷O, ¹⁵N, ¹³C, ²H and combinations thereof.

Detecting and quantifying the amount of the UCK2 fragment peptide in the protein digest may indicate the presence of modified or unmodified UCK2 protein and an association with cancer in the subject. The results of the detecting and/or quantifying the amount of the UCK2 fragment peptide, or the level of the UCK2 protein may be correlated to the diagnostic stage/grade/status of the cancer. Correlating the results of the detecting and/or quantifying the amount of the UCK2 fragment peptide, or the level of the UCK2 protein to the diagnostic stage/grade/status of the cancer may be combined with detecting and/or quantifying the amount of other proteins or peptides from other proteins in a multiplex format to provide additional information about the diagnostic stage/grade/status of the cancer.

In a further step, the patient from which the biological sample was obtained may be administered a therapeutically effective amount of a therapeutic agent, where the therapeutic agent and/or amount of the therapeutic agent administered is based upon the amount of the UCK2 fragment peptide or the level of UCK2 protein. The therapeutic agent may bind the UCK2 protein and/or inhibits its biological activity.

Also provided is a method of treating a patient suffering from cancer, such as colon cancer, comprising:

(a) quantifying the level of a specified UCK2 fragment peptide in a protein digest prepared from a tumor sample obtained from the patient and calculating the level of the UCK2 peptide in the sample by selected reaction monitoring using mass spectrometry;

(b) comparing the level of the UCK2 fragment peptide to a reference level, and

(c) treating the patient with FOLFOX chemotherapy regimen when the level of the UCK2 fragment peptide is higher than the reference level or

(d) treating the patient with a therapeutic regimen that does not comprise the FOLFOX chemotherapy regimen when the level of the UCK2 fragment peptide is below the reference level.

The reference level may be 319 amol/μg., +/−250 amol/μg, +/−150 amol/μg, +/−100 amol/μg, +/−50 amol/μg, or +/−25 amol/μg, of biological sample protein analyzed.

The protein digest may be a protease digest, such as a trypsin digest.

The mass spectrometry may be tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry, and the mode of mass spectrometry used may be Selected Reaction Monitoring (SRM), Multiple Reaction Monitoring (MRM), and/or multiple Selected Reaction Monitoring (mSRM).

The tumor sample may be a cell, collection of cells, or a solid tissue, and may be formalin fixed solid tissue. The tissue may be paraffin embedded tissue.

Detecting and quantitating the specified UCK2 fragment peptide can be combined with detecting and quantitating other peptides from other proteins in multiplex so that the treatment decision about which agent used for treatment is based upon specific levels of the specified UCK2 fragment peptide in combination with other peptides/proteins in the biological sample.

### BRIEF DESCRIPTION OF THE DRAWING

FIG. 1 provides Overall Survival (OS) and Relapse-free Survival (RFS) curves showing that patients whose tumor tissue express levels of the UCK2 protein>319 amol/ug of tumor cell protein have statistically significantly longer survival than patients whose tumor cells express<319 amol/ug of tumor cell protein when treated with the combinatorial FOLFOX (5-fluorouracil, folinic acid, and oxaliplatin) chemotherapy regimen. Results are shown as probability (0-100%) for OS and RFS in days after initiation of treatment in the Kaplan-Meier curve. The Mantel-Cox log-rank test was used to determine that these results are statistically significant. Hazard Ratio (HR) for OS=0.3484 with a 95% CI=0.1339 to 0.9063; HR for RFS=0.4483 with a 95% CI=0.1994 to 1.008.

### DETAILED DESCRIPTION

UCK2 (also known as uridine-cytidine kinase 2) is an enzyme that catalyzes the phosphorylation of uridine and cytidine to uridine monophosphate (UMP) and cytidine monophosphate (CMP), respectively. This is the first step in the production of the pyrimidine nucleoside triphosphates required for RNA and DNA synthesis. UCK2 has been shown to be an integral part of the fluoropyrimidine pathway and, when highly expressed in a tumor cell, it promotes greater sensitivity to treatment with 5-FU, whereas lower to no expression helps confer resistance of tumor cells to treatment with 5-FU. Accordingly, the presence and quantitative levels of UCK2 are relevant to determining the probability that a tumor cell may be sensitive or resistant to treatment with 5-FU and so it would be very informative to the cancer treatment decision process to know the quantitative amount of the UCK2 protein in patient tumor cells.

The methods below provide improved methods of treatment for cancer patients using a quantitative proteomics-based assay that quantifies the UCK2 protein in formalin fixed tissues from the patients. Data from this assay are used to inform improved treatment decisions for cancer therapy by, for example, predicting response to the chemotherapy regimen FOLFOX commonly used in treating colon cancer.

The mass spectrometry-based UCK2 SRM assay can be used to measure relative or absolute quantitative levels of UCK2 in a given protein preparation obtained from a biological sample. More specifically, the SRM assay can measure UCK2 directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by references in their entirety. The methods described in U.S. Pat. No. 7,473,532 may conveniently be carried out using Liquid Tissue® reagents and protocol available from Expression Pathology Inc. (Rockville, Md.).

The most widely and advantageously available form of tissue from cancer patients is formalin fixed, paraffin embedded tissue. Formaldehyde/formalin fixation of surgically removed tissue is far and away the most common method of preserving cancer tissue samples worldwide and is the accepted convention for standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, added to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from the presently described SRM assay can be used to correlate accurate and precise quantitative levels of UCK2 within the specific tissue samples (e.g., cancer tissue sample) of the patient or subject from whom the tissue (biological sample) was collected and preserved. This not only provides diagnostic information about the cancer, but also permits a physician or other medical professional to determine appropriate therapy for the patient. Such an assay that provides diagnostically and therapeutically important information about levels of protein expression in a diseased tissue or other patient sample is termed a companion diagnostic assay. For example, such an assay can be designed to diagnose the stage or degree of a cancer and determine a therapeutic agent to which a patient is most likely to respond.

The presently assay described herein measures absolute levels of a specified unmodified UCK2 peptide. An absolute quantitative level of the UCK2 protein is determined by, for example, the SRM methodology whereby the SRM signature peak area of an individual peptide from the designated protein in a biological sample is compared to the SRM signature peak area of a spiked internal standard. In one embodiment, the internal standard is a synthetic version of the peptide with the same amino acid sequence derived from the designated protein that contains one or more amino acid residues labeled with one or more heavy isotopes. Such isotope-labeled internal standards are synthesized so that, when analyzed by mass spectrometry, a standard generates a predictable and consistent SRM signature peak that is different and distinct from the native peptide signature peak and which can be used as a comparator peak. Thus, when the internal standard is spiked into a protein preparation from a biological sample in known amounts and analyzed by mass spectrometry, the SRM signature peak area of the native peptide is compared to the SRM signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

The SRM assay method can be used to aid diagnosis of the stage of cancer, for example, directly in patient-derived tissue, such as formalin fixed tissue, and to aid in determining which therapeutic agent would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not a specific protein, or proteins, and which forms of proteins, are present in that patient tissue. Moreover, the expression level of a protein, or multiple proteins, can be determined and compared to a “normal” or reference level found in healthy tissue. Normal or reference levels of proteins found in healthy tissue may be derived from, for example, the relevant tissues of one or more individuals that do not have cancer. Alternatively, normal or reference levels may be obtained for individuals with cancer by analysis of relevant tissues not affected by the cancer.

Assay of protein levels from one or more proteins can also be used to diagnose the stage of cancer in a patient or subject diagnosed with cancer by employing the protein levels. The level of an individual peptide derived from a protein is defined as the molar amount of the peptide determined by the SRM assay per total amount of protein lysate analyzed. Information regarding a designated protein or proteins can thus be used to aid in determining the stage or grade of a cancer by correlating the level of the protein(s) (or fragment peptides from the proteins) with levels observed in normal tissues. Once the quantitative amount of one or more proteins has been determined in the cancer cells, that information can be matched to a list of therapeutic agents (chemical and biological) developed to specifically treat cancer tissue that is characterized by, for example, abnormal expression of the protein or protein(s) that were assayed. Matching information from a protein assay to a list of therapeutic agents that specifically targets, for example, the designated protein or cells/tissue expressing the protein, defines what has been termed a personalized medicine approach to treating disease. The assay methods described herein form the foundation of a personalized medicine approach by using analysis of proteins from the patient's own tissue as a source for diagnostic and treatment decisions.

In principle, any predicted peptide derived from a designated protein, prepared for example by digesting with a protease of known specificity (e.g. trypsin), can be used as a surrogate reporter to determine the abundance of a designated protein in a sample using a mass spectrometry-based SRM assay. Similarly, any predicted peptide sequence containing an amino acid residue at a site that is known to be potentially modified in the designated protein also might potentially be used to assay the extent of modification of the designated protein in a sample.

Suitable fragment peptides derived from the UCK2 protein may be generated by a variety of means including by the use of the Liquid Tissue® protocol provided in U.S. Pat. No. 7,473,532. The Liquid Tissue® protocol and reagents are capable of producing peptide samples suitable for mass spectroscopic analysis from formalin fixed paraffin embedded tissue by proteolytic digestion of the proteins in the tissue/biological sample. In the Liquid Tissue® protocol the tissue/biological is heated in a buffer for an extended period of time (e.g., from about 80° C. to about 100° C. for a period of time from about 10 minutes to about 4 hours) to reverse or release protein cross-linking. The buffer employed is a neutral buffer, (e.g., a Tris-based buffer, or a buffer containing a detergent). Following heat treatment the tissue/biological sample is treated with one or more proteases, including but not limited to trypsin, chymotrypsin, pepsin, and endoproteinase Lys-C for a time sufficient to disrupt the tissue and cellular structure of said biological sample. The result of the heating and proteolysis is a liquid, soluble, dilutable biomolecule lysate.

Surprisingly, it was found that many potential peptide sequences from the UCK2 protein are unsuitable or ineffective for use in mass spectrometry-based SRM assays for reasons that are not immediately evident. As it was not possible to predict the most suitable peptides for an individual SRM assay, it was necessary to experimentally identify modified and unmodified peptides in actual Liquid Tissue® lysates to develop a reliable and accurate SRM assay for the UCK2 protein. While not wishing to be bound by any theory, it is believed that some peptides might, for example, be difficult to detect by mass spectrometry because they do not ionize well or produce fragments distinct from other proteins. Peptides may also fail to resolve well in separation (e.g., liquid chromatography), or may adhere to glass or plastic ware.

The most optimal peptide for n UCK2 SRM assay was derived by protease digestion of all the proteins within a complex Liquid Tissue® lysate prepared from cells procured from formalin fixed cancer tissue. Unless noted otherwise, in each instance the protease was trypsin. The Liquid Tissue® lysate was then analyzed by mass spectrometry to determine those peptides derived from the UCK2 protein that are detected and analyzed by mass spectrometry. Identification of a specific preferred subset of peptides for mass spectrometric analysis is based on; 1) experimental determination of which peptide or peptides from a protein ionize in mass spectrometry analyses of Liquid Tissue® lysates, and 2) the ability of the peptide to survive the protocol and experimental conditions used in preparing a Liquid Tissue® lysate. This latter property extends not only to the amino acid sequence of the peptide but also to the ability of a modified amino acid residue within a peptide to survive in modified form during the sample preparation.

Protein lysates from cells procured directly from formalin (formaldehyde)-fixed tissue were prepared using the Liquid Tissue® reagents and protocol that comprises collecting cells into a sample tube via tissue microdissection followed by heating the cells in the Liquid Tissue® buffer for an extended period of time. Once the formalin-induced cross linking had been negatively affected, the tissue/cells were then digested to completion in a predictable manner using a protease, such as trypsin. Each protein lysate was reduced to a collection of peptides by digestion of intact polypeptides with the protease. Each Liquid Tissue® lysate was analyzed (e.g., by ion trap mass spectrometry) to perform multiple global proteomic surveys of the peptides where the data was presented as identification of as many peptides as could be identified by mass spectrometry from all cellular proteins present in each protein lysate. An ion trap mass spectrometer or another form of a mass spectrometer that is capable of performing global profiling for identification of as many peptides as possible from a single complex protein/peptide lysate is typically employed. Ion trap mass spectrometers however may be the best type of mass spectrometer for conducting global profiling of peptides.

Although an SRM assay can be developed and performed on any type of mass spectrometer, including a MALDI, ion trap, or triple quadrupole, the most advantageous instrument platform for an SRM assay is often considered to be a triple quadrupole instrument platform. Once as many UCK2 peptides as possible were identified in a single MS analysis of a single lysate under the conditions employed, then that list of peptides was collated and used to determine which UCK2 peptides were detected in that lysate. That process was repeated for multiple Liquid Tissue lysates, and the list of peptides was collated into a single dataset in order to identify the most optimal fragment peptide for the presently described assay. The most optimal tryptic peptide that defines the presently described assay was found to be LFVDTDADTR identified as SEQ ID NO:1 in Table 1. This peptide was optimally detected from multiple Liquid Tissue® lysates of multiple different formalin fixed tissues of different human organs including, for example, prostate, colon, and breast.

TABLE 1

| SEQ ID | Peptide Sequence |
|---|---|
| SEQ ID NO: 1 | LFVDTDADTR |

One consideration when conducting an SRM assay is the type of instrument that maybe employed in the analysis of the peptides. Although SRM assays can be developed and performed on any type of mass spectrometer, including a MALDI, ion trap, or triple quadrupole, the most advantageous instrument platform for an SRM assay is often considered to be a triple quadrupole instrument platform. That type of a mass spectrometer may be considered to be the most suitable instrument for analyzing a single isolated target peptide within a very complex protein lysate that may consist of hundreds of thousands to millions of individual peptides from all the proteins contained within a cell.

Assessment of corresponding protein levels in tissues based on analysis of formalin fixed patient-derived tissue can provide diagnostic, prognostic, and therapeutically-relevant information about each particular patient. This disclosure describes a method for measuring the level of the UCK2 protein in a biological sample, comprising detecting and/or quantifying the amount of the specified UCK2 fragment peptide in a protein digest prepared from said biological sample using mass spectrometry; and calculating the level of UCK2 protein in said sample; and wherein said level is a relative level or an absolute level. In a related embodiment, quantifying a specified fragment peptide comprises determining the amount of the fragment peptide in a biological sample by comparison to an added internal standard peptide of known amount, wherein the fragment peptide in the biological sample is compared to an internal standard peptide having the same amino acid sequence. In some embodiments the internal standard is an isotopically labeled internal standard peptide comprises one or more heavy stable isotopes selected from ¹⁸0, ¹⁷0, ³⁴S, ¹⁵N, ¹³C, ²H or combinations thereof.

The UCK2 protein is involved in the metabolic process of the fluoropyrimidine pathway and when highly expressed in a tumor cell promotes greater sensitivity to treatment with 5-FU whereas lower to no expression helps confer resistance of tumor cells to treatment with 5-FU. Thus the presence and quantitative levels of UCK2 has relevance in determining the probability that a tumor cell may be sensitive or resistant to treatment with 5-FU.

The method for measuring the level of UCK2 protein in a biological sample described herein may be used as a diagnostic indicator of cancer in a patient or subject, and an indicator of the most optimal therapy for that patient or subject. In one embodiment, the results from measurements of the level of UCK2 protein may be employed to determine whether or not a cancer patient will respond to treatment comprising the combinatorial FOLFOX (5-fluorouracil, folinic acid, and oxaliplatin) regimen. Tumor tissue from the patient is assayed using the methods described above and the level of expression of UCK2 in that tissue is measured. If the measured UCK2 expression is above the reference level then the patient is treated with FOLFOX according to accepted clinical protocols. If the measured UCK2 expression is below the reference level the patient is treated with an alternative therapeutic regimen. Alternative treatment regimens include use of the drugs trifluridine, irinotecan, cetuximab, and/or panitumumab. For example, the patient may be treated with a combination of irinotecan plus cetuximab using well known clinical protocols.

Because both nucleic acids and protein can be analyzed from the same Liquid Tissue® biomolecular preparation it is possible to generate additional information about disease diagnosis and drug treatment decisions from the nucleic acids in same sample upon which UCK2 was analyzed. For example, if the UCK2 protein is expressed by certain cells at increased levels, when assayed by SRM the data can provide information about the state of the cells and their potential for uncontrolled growth, potential drug resistance/sensitivity, and the development of other cancers. At the same time, information about the status of the corresponding genes and/or the nucleic acids and proteins they encode (e.g., mRNA molecules and their expression levels or splice variations) can be obtained from nucleic acids present in the same Liquid Tissue biomolecular preparation that can be assessed simultaneously to the UCK2 SRM analysis. Any gene and/or nucleic acid that does not code for the designated protein and which is present in the same biomolecular preparation can be assessed simultaneously to UCK2 SRM analysis. In one embodiment, information about the UCK2 protein and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

The presently described method demonstrates the ability to determine which colon cancer patients will likely show a longer overall survival as measured by days when treated with the combinatorial FOLFOX (5-fluorouracil, folinic acid, and oxaliplatin) cancer treatment regimen.

### EXAMPLE

### Determination of a Predictive Value of UCK2 Protein Expression for Combination Therapy Comprising the FOLFOX (5-Fluorouracil, Folinic Acid, and Oxaliplatin) Cancer Treatment Regimen in a Population of Colon Cancer Patients

### Patients:

128 stage II/stage III patients were identified with colorectal cancer (CRC). Tumors were surgically removed prior to treatment and archived as formalin-fixed, paraffin-embedded (FFPE) tissue and all were histologically confirmed as CRC. All patients were treated with adjuvant FOLFOX (5-fluorouracil, folinic acid, and oxaliplatin) treatment regimen for up to 12 cycles.

Methods: Archived formalin-fixed, paraffin-embedded tissue sections were obtained from patients with stage II/stage III colon cancer that were treated with FOLFOX (n=128). A board-certified pathologist marked the tumor areas, which were microdissected and the tissue was solubilized using the Liquid Tissue® protocol and reagents. In each liquefied tumor sample, 60 protein biomarkers including UCK2 were quantified with selected reaction monitoring mass spectrometry. Patients were stratified by a UCK2 cutoff of 319 amol/ug, using standard statistical methods to derive the most significant cutoff value for overall survival. Survival outcomes were assessed with Kaplan-Meier and Mantel-Cox log-rank analyses.

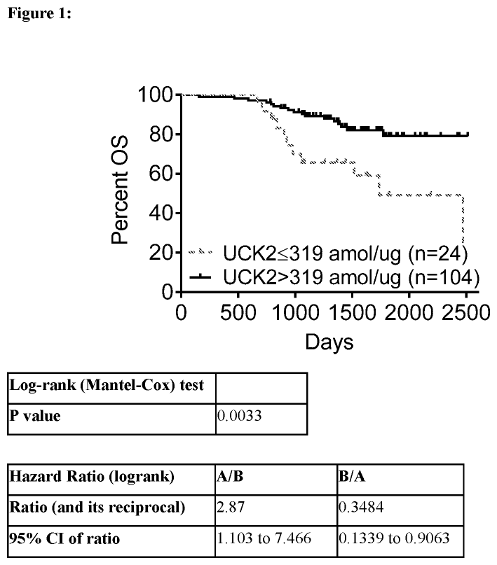

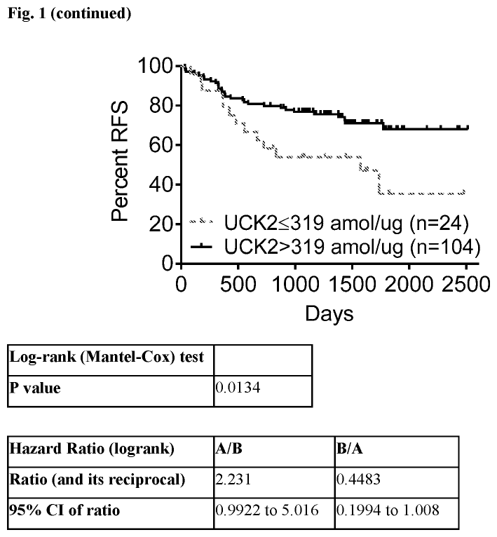

Results: Among 128 FOLFOX-treated colon cancer patients, those with UCK2 protein levels above the cutoff of 319 amol/ug (n=104) had better overall survival (OS) (HR: 0.3484; 95% CI: 0.1339-0.9063; p=0.0033) and relapse-free survival (RFS) (HR: 0.4483; 95% CI: 0.1994-1.008; p=0.0134) than patients with UCK2 levels below the cutoff (n=24). Data from the analyses is shown in FIG. 1.

Conclusions: Mass spectrometric evaluation of UCK2 retrospectively identified responders to FOLFOX chemotherapy and could be used to predict response in colon cancer patients. Multiplexed proteomics can quantitate UCK2 simultaneously with other therapeutically relevant proteins (e.g., HER2, ALK, ROS1) to inform therapy selection at initial diagnosis and upon relapse.

1. A method for measuring a level of UCK2 protein in a human biological sample of formalin-fixed tissue, the method comprising
detecting and quantifying an amount of a UCK2 fragment peptide in a protein digest prepared from said human biological sample using mass spectrometry; and
calculating the level of UCK2 protein in said sample; wherein the UCK2 fragment peptide is SEQ ID NO:1.

2. The method of claim 1, further comprising the step of fractionating said protein digest prior to detecting and quantifying the amount of said UCK2 fragment peptide.

3. The method of claim 1, wherein said protein digest comprises a protease digest.

4. The method of claim 1, wherein the tissue is paraffin-embedded tissue.

5. The method of claim 1, wherein the tissue is obtained from a tumor.

6. The method of claim 1, wherein quantifying said UCK2 fragment peptide comprises comparing the amount of said UCK2 fragment peptide in the human biological sample to the an amount of the same UCK2 fragment peptide in a different and separate biological sample.

7. The method of claim 1, wherein quantifying said UCK2 fragment peptide comprises comparing said UCK2 fragment peptide in the human biological sample to an internal standard peptide having the same amino acid sequence; and wherein the internal standard peptide is an isotopically labeled peptide.

8. The method of claim 7, wherein the isotopically labeled internal standard peptide comprises one or more heavy stable isotopes selected from ¹⁸O, ¹⁷O, ¹⁵N, ¹³C, ²H and a combination thereof.

9. The method of claim 1, wherein detecting and quantifying the amount of said UCK2 fragment peptide in the protein digest indicates the presence of UCK2 protein and an association with cancer in a subject.

10. The method of claim 9, further comprising correlating results of said detecting and quantifying the amount of said UCK2 fragment peptide, or the level of said UCK2 protein to the diagnostic stage/grade/status of the cancer.

11. The method of claim 10, wherein correlating the results of said detecting and quantifying the amount of said UCK2 fragment peptide, or the level of said UCK2 protein to the diagnostic stage/grade/status of the cancer is combined with detecting and/or quantifying the amount of other proteins or peptides from other proteins in a multiplex format.

12. The method of claim 1, further comprising administering to a patient from which said biological sample was obtained a therapeutically effective amount of a therapeutic agent, wherein the therapeutic agent and/or amount of the therapeutic agent administered is based upon the amount of said UCK2 fragment peptide or the level of UCK2 protein.

13. The method of claim 12, wherein said therapeutic agent binds the UCK2 protein and/or inhibits its biological activity.

14. A method of treating a patient suffering from cancer, comprising:
(a) quantifying a level of a UCK2 fragment peptide in a protein digest prepared from a tumor sample obtained from the patient and calculating the level of the UCK2 peptide in said sample by selected reaction monitoring using mass spectrometry;
(b) comparing the level of said UCK2 fragment peptide to a reference level, and
(c) treating the patient with FOLFOX chemotherapy regimen when the level of the UCK2 fragment peptide is higher than said reference level or
(d) treating the patient with a therapeutic regimen that does not comprise the FOLFOX chemotherapy regimen when the level of the UCK2 fragment peptide is below said reference level.

15. The method of claim 14, wherein said patient is suffering from colon cancer.

16. The method of claim 14, wherein said reference level is 319 amol/μg., +/−250 amol/μg, +/−150 amol/μg, +/−100 amol/μg, +/−50 amol/μg, or +/−25 amol/μg of tumor sample protein analyzed.

17.-20. (canceled)

21. The method of claim 14, wherein said protein digest comprises a trypsin digest.

22. The method of claim 14, wherein the mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

23. The method of claim 22, wherein a mode of mass spectrometry used is Selected Reaction Monitoring (SRM), Multiple Reaction Monitoring (MRM), and/or multiple Selected Reaction Monitoring (mSRM).

24. The method of claim 14, wherein the tumor sample is a cell, collection of cells, or a solid tissue.

25. The method of claim 24, wherein the tumor sample is formalin fixed solid tissue.

26. The method of claim 25, wherein the tissue is paraffin embedded tissue.

27. The method of claim 14, wherein detecting and quantitating the UCK2 fragment peptide is combined with detecting and quantitating other peptides from other proteins in a multiplex format.

28. The method of claim 3, wherein the protease digest is a trypsin digest.

29. The method of claim 1, wherein the mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

30. The method of claim 29, wherein a mode of mass spectrometry used is Selected Reaction Monitoring (SRM), Multiple Reaction Monitoring (MRM), and/or multiple Selected Reaction Monitoring (mSRM).

31. The method of claim 1, wherein the tumor sample is a cell, collection of cells, or a solid tissue.

32. The method of claim 31, wherein the tumor sample is formalin fixed solid tissue.

33. The method of claim 32, wherein the tissue is paraffin embedded tissue.
